(19) United States
(12) Patent Application Publication
(10) Pub. No.: US 2016/0074598 A1
(43) Pub. Date: Mar. 17, 2016

(54) SAFETY SYRINGE

(71) Applicant: Hsien-Tsung WANG, Taipei (TW)

(72) Inventor: Hsien-Tsung WANG, Taipei (TW)

(21) Appl. No.: 14/536,695

(22) Filed: Nov. 10, 2014

(30) Foreign Application Priority Data
Sep. 17, 2014 (TW) 103132113
Sep. 17, 2014 (TW) 103216521

(51) Int. Cl. A61M 5/32

(52) U.S. Cl. CPC A61M 5/3216

(57) ABSTRACT

A safety syringe is provided with a barrel including a casing tube and a hollow, cylindrical tip; a needle assembly releasably secured to the tip; a pivot assembly releasably secured to the barrel; and a locking cover assembly pivotably secured to the pivot assembly. In inoperative position, the locking cover assembly is distal the needle assembly which is exposed. After dispensing, a pivotal movement of the locking cover assembly toward the needle assembly is configured to cover and lock the needle. This can prevent the needle from accidentally pricking a medical employee to contaminate the medical employee with microorganisms or blood on the needle. Further, this can prevent the safety syringe from being used for a second time. Thus, the safety syringe is ready for disposal.

### BACKGROUND OF THE INVENTION

1. Field of the Invention

The invention relates to hypodermic syringes and more particularly to a safety syringe with a tamper-proof characteristic.

2. Description of Related Art

A major cause of the spread of infectious disease in the population is due to the presence of exposed hypodermic syringes. Infection can be spread from accidental needle sticks by way of exposed, contaminated needles used in high risk patients in hospitals and medical facilities. Used syringes with extended needles present a risk to medical employees and others.

Conventional safety syringes are characterized by that the exposed needles will retract at the end of the injection cycle. Further, some prior safety syringes are characterized by that the used needle is retracted in an internal concealed position and cannot be reused (i.e., being self-destructed and single use).

While a wide range of safety syringes are disclosed, the need for advancement of the art still exists.

### SUMMARY OF THE INVENTION

It is therefore one object of the invention to provide a safety syringe having a hypodermic needle which can be concealed after use. This can prevent the needle from accidentally pricking a medical employee to contaminate the medical employee with microorganisms or blood on the needle. Further, this can prevent the safety syringe from being used for a second time.

For achieving above and other objects, in a first aspect of the invention there is provided a barrel including a casing tube and a hollow, cylindrical tip; a needle assembly releasably secured to the tip; a pivot assembly releasably secured to the barrel; and a locking cover assembly pivotably secured to the pivot assembly; wherein in inoperative position, the locking cover assembly is distal the needle assembly which is exposed; and wherein in response to a dispensing operation of the needle assembly, a pivotal movement of the locking cover assembly toward the needle assembly is configured to cover and lock the needle assembly.

Preferably, the tip includes internal threads on an inner surface; the needle assembly includes two opposite protuberances on a rear end, the protuberances being threadedly secured to the threads so as to fasten the needle assembly and the tip of the barrel together; the pivot assembly is secured to the tip and includes an enlarged latch formed at an end of the neck, and a slot formed on the latch; and the locking cover assembly is releasably secured to the neck.

Preferably, the locking cover assembly includes a grooved body having an extension, and a pivot hole through an end of the extension; a groove formed in the grooved body; and at least one of first and second locking members formed on at least one side of the groove; and wherein the pivot hole is pivotably fastened on the neck.

Preferably, the locking cover assembly further comprising with two opposite splay members at an end of the groove proximate the extension.

Preferably, the needle assembly is mounted on the tip; wherein the pivot assembly is mounted on a periphery of a front end of the casing tube and includes a central trough having a C-shaped longitudinal section and an opening distal the casing tube; and wherein the locking cover assembly is pivotably secured to the central trough.

Preferably, the tip includes internal threads on an inner surface; the needle assembly includes two opposite protuberances on a rear end, the protuberances being threadedly secured to the threads so as to fasten the needle assembly and the tip of the barrel together; the pivot assembly is secured to the tip and includes a central trough having a C-shaped longitudinal section and an opening distal the casing tube; and the locking cover assembly is pivotably secured to the central trough.

Preferably, the locking cover assembly includes a grooved body having an extension, a shaft formed across two sides of an inner surface at an end of the extension, a groove formed in the grooved body, and at least one of first and second locking members formed on at least one side of the groove; and wherein the shaft is pivotably fastened in the trough.

Preferably, the locking cover assembly further comprising with two opposite splay members at an end of the groove proximate the extension.

In a second aspect of the invention there is provided an arrangement for a barrel including a casing tube and a hollow, cylindrical tip; a needle assembly releasably secured to the tip; a pivot assembly secured to the needle assembly; and a locking cover assembly pivotably secured to the pivot assembly; wherein in inoperative position, the locking cover assembly is distal the needle assembly which is exposed; and wherein in response to a dispensing operation of the needle assembly, a pivotal movement of the locking cover assembly toward the needle assembly is configured to cover and lock the needle assembly.

Preferably, the pivot assembly includes a base formed with the needle assembly, an enlargement at an end, and a cylindrical neck having a diameter less than that of each of the base and the enlargement; and wherein the locking cover assembly is releasably secured to the neck.

Preferably, the locking cover assembly includes a grooved body having an extension, and a pivot hole through an end of the extension; a groove formed in the grooved body; and at least one of first and second locking members formed on at least one side of the groove; and wherein the pivot hole is pivotably fastened on the neck by passing through the enlargement.

Preferably, the locking cover assembly further comprising with two opposite splay members at an end of the groove proximate the extension.

Preferably, the pivot assembly includes a central trough having a C-shaped longitudinal section and an opening distal the casing tube; and wherein the locking cover assembly is pivotably secured to the central trough.

Preferably, the locking cover assembly includes a grooved body having an extension, a shaft formed across two sides of an inner surface at an end of the extension, a groove formed in the grooved body, and at least one of first and second locking members formed on at least one side of the groove; and wherein the shaft is pivotably fastened in the trough.

Preferably, the locking cover assembly further comprising with two opposite splay members at an end of the groove proximate the extension.

In a third aspect of the invention there is provided an arrangement for a barrel including a casing tube and a hollow, cylindrical tip; an adapter releasably secured to the tip; a needle assembly releasably secured to the adapter; a pivot assembly secured to the adapter; and a locking cover assembly pivotably secured to the adapter; wherein in inoperative position, the locking cover assembly is distal the needle assembly which is exposed; and wherein in response to a dispensing operation of the needle assembly, a pivotal movement of the locking cover assembly toward the needle assembly is configured to cover and lock the needle assembly.

Preferably, the adapter is hollow, elongated and includes a larger diameter section proximate the barrel, and a smaller diameter section with the needle assembly lockingly put thereon; wherein the pivot assembly includes a base formed with the larger diameter section, an enlargement at an end, and a neck between the base and the enlargement; and wherein the locking cover assembly is releasably secured to the neck.

Preferably, the tip includes internal threads on an inner surface; the adapter includes a larger diameter section proximate the barrel, a smaller diameter section secured to the needle assembly, and two opposite protuberances on a rear end, the protuberances being threadedly secured to the threads so as to fasten the adapter and the tip of the barrel together; the pivot assembly includes a base formed with the larger diameter section, an enlargement at an end, and a neck between the base and the enlargement; the smaller diameter section is formed with the needle assembly; and the locking cover assembly is releasably secured to the neck.

Preferably, the locking cover assembly includes a grooved body having an extension, and a pivot hole through an end of the extension; a groove formed in the grooved body; and at least one of first and second locking members formed on at least one side of the groove; and wherein the pivot hole is pivotably fastened on the neck.

Preferably, the locking cover assembly further comprising with two opposite splay members at an end of the groove proximate the extension.

Preferably, the adapter is hollow, cylindrical and fastened on the tip; wherein the pivot assembly is mounted on a periphery of the adapter and includes a central trough having a C-shaped longitudinal section and an opening distal the casing tube; and wherein the locking cover assembly is pivotably secured to the central trough.

Preferably, the locking cover assembly includes a grooved body having an extension, a shaft formed across two sides of an inner surface at an end of the extension, a groove formed in the grooved body, and at least one of first and second locking members formed on at least one side of the groove; and wherein the shaft is pivotably fastened in the trough.

Preferably, the locking cover assembly further comprising with two opposite splay members at an end of the groove proximate the extension.

The above and other objects, features and advantages of the invention will become apparent from the following detailed description taken with the accompanying drawings.

### BRIEF DESCRIPTION OF THE DRAWINGS

FIG. 1 is a perspective view of a safety syringe according to a first preferred embodiment of the invention;

FIG. 2 is a view similar to FIG. 1 with both the needle assembly and the locking cover assembly detached;

FIG. 3 is a view similar to FIG. 1 with the locking cover assembly pivoted to cover the needle assembly after use and prior to disposal;

FIG. 4 is a top view of FIG. 1 in part section;

FIG. 5 is a top view of FIG. 3 in part section;

FIG. 6 is a perspective view of the second configuration of the locking cover assembly of the safety syringe according to the first preferred embodiment;

FIG. 7 is a perspective view of another configuration of the locking cover assembly shown in FIG. 6;

FIG. 8 is a perspective view of a safety syringe according to a second preferred embodiment of the invention;

FIG. 9 is a view similar to FIG. 8 with both the locking cover assembly and the needle assembly detached;

FIG. 10 is a view similar to FIG. 8 with the locking cover assembly pivoted to cover the needle assembly after use and prior to disposal;

FIG. 11 is an enlarged view of the needle assembly covered by the locking cover assembly after use;

FIG. 12 is a perspective view of the safety syringe of the second preferred embodiment incorporating a second configuration of the locking cover assembly;

FIG. 13 is a perspective view of the locking cover assembly shown in FIG. 12 with the needle assembly being covered by the locking cover assembly;

FIG. 14 is a perspective view of a safety syringe according to a third preferred embodiment of the invention;

FIG. 15 is an exploded view of the safety syringe in FIG. 14;

FIG. 16 is a perspective view of the safety syringe of the third preferred embodiment incorporating a second configuration of the locking cover assembly;

FIG. 17 is a perspective view of a safety syringe according to a fourth preferred embodiment of the invention;

FIG. 18 is an exploded perspective view of the safety syringe of FIG. 17;

FIG. 19 is another exploded perspective view in part section of the safety syringe of FIG. 18;

FIG. 20 is a view similar to FIG. 17 with the locking cover assembly pivoted to cover the needle assembly after use and prior to disposal;

FIG. 21 is a perspective view of the second configuration of the locking cover assembly and the needle assembly of the safety syringe according to the fourth preferred embodiment;

FIG. 22 is a perspective view of another configuration of the locking cover assembly shown in FIG. 21;

FIG. 23 is a perspective view of a safety syringe according to a fifth preferred embodiment of the invention;

FIG. 24 is an exploded perspective view of the safety syringe in FIG. 23;

FIG. 25 is a perspective view of the safety syringe of the fifth preferred embodiment incorporation a second configuration of the locking cover assembly;

FIG. 26 is a perspective view of a safety syringe according to a sixth preferred embodiment of the invention;

FIG. 27 is an exploded perspective view of the safety syringe in FIG. 26;

FIG. 28 is a perspective view of the safety syringe of the sixth preferred embodiment incorporation a second configuration of the locking cover assembly;

FIG. 29 is a perspective view of a safety syringe according to a seventh preferred embodiment of the invention;

FIG. 30 is an exploded view of the safety syringe in FIG. 29;

FIG. 31 is a perspective view of the safety syringe of the seventh preferred embodiment incorporation a second configuration of the locking cover assembly;

FIG. 32 is a perspective view of a safety syringe according to an eighth preferred embodiment of the invention;

FIG. 33 is an exploded perspective view of the safety syringe in FIG. 32; and

FIG. 34 is a perspective view of the safety syringe of the eighth preferred embodiment incorporation a second configuration of the locking cover assembly.

### DETAILED DESCRIPTION OF THE INVENTION

The invention is directed to a safety syringe comprising a barrel including a casing tube and a hollow, cylindrical tip; a needle assembly releasably secured to the tip; a pivot assembly releasably secured to the barrel; and a cover assembly pivotably secured to the pivot assembly. In inoperative position, the locking cover assembly is distal the needle assembly which is exposed. After dispensing, a pivotal movement of the locking cover assembly toward the needle assembly is configured to cover and lock the needle. This can prevent the needle from accidentally pricking a medical employee to contaminate the medical employee with microorganisms or blood on the needle. Further, this can prevent the safety syringe from being used for a second time. Thus, the safety syringe is ready for disposal.

In the invention, the pivot assembly discussed below is comprehensive. Specifically, it covers any pivotal arrangement mounted on the syringe, the needle assembly, or the adapter, and the corresponding pivot hole formed on the locking cover assembly. More specifically, it covers any pivotal arrangement having a C-shaped longitudinal section mounted on the syringe, the needle assembly, or the adapter, and the corresponding pivot. The pivot assembly is pivotably secured to the pivot hole and the pivotal arrangement is pivotably secured to the pivot.

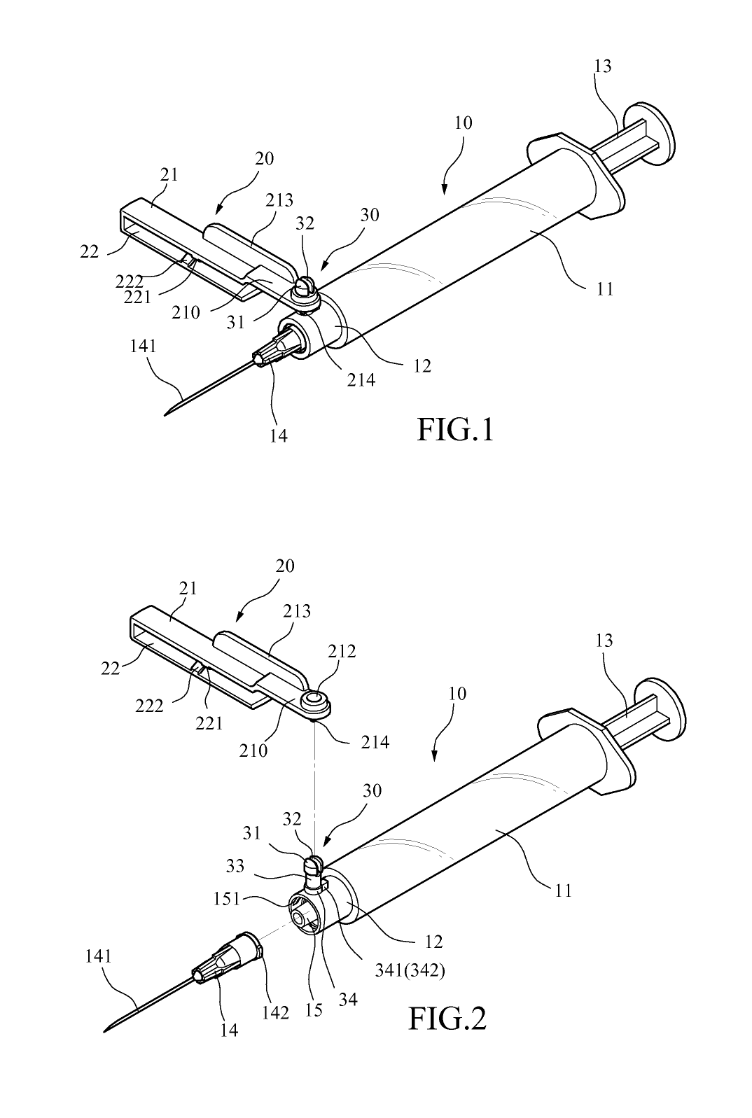

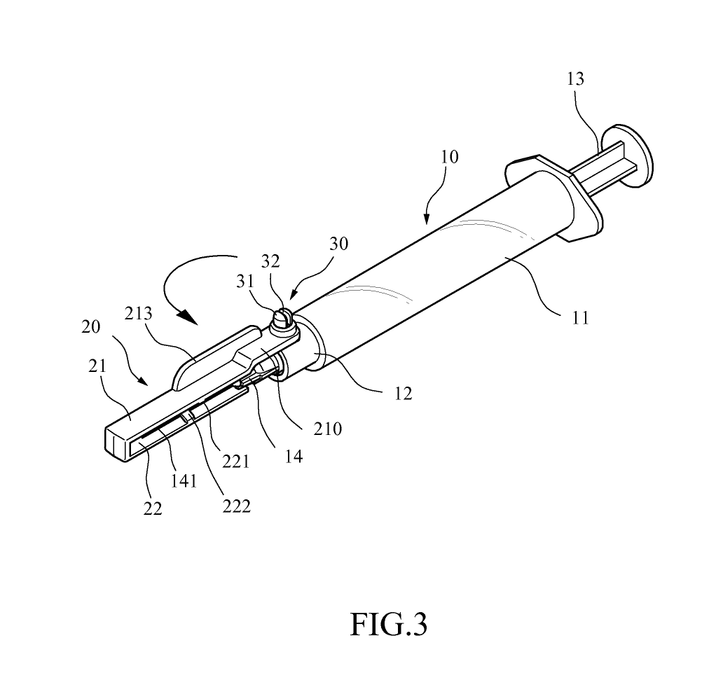

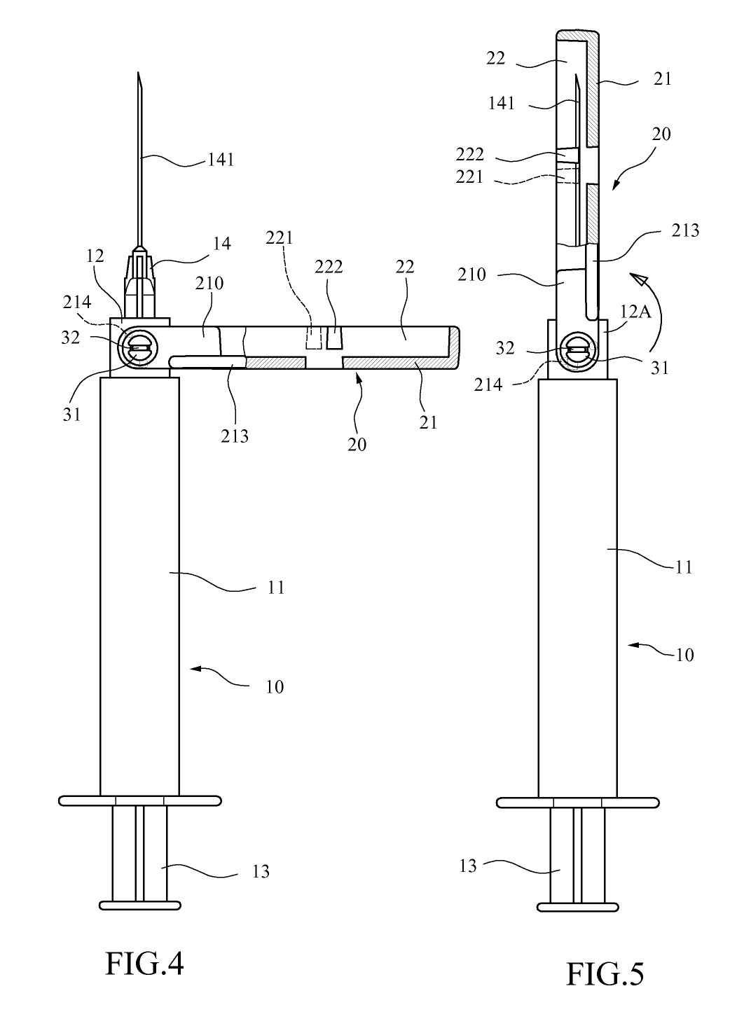

Referring to FIGS. 1 to 7, a safety syringe in accordance with a first preferred embodiment of the invention comprises a barrel 10, a locking cover assembly 20, and a pivot assembly 30 as discussed in detail below.

The barrel 10 includes a cylindrical casing tube 11, a cylindrical tip 12 having a diameter less than that of the casing tube 11, and a stem 13. A needle assembly 14 has a hypodermic needle 141 and two opposite protuberances 142.

A hollow cylinder 15 is concentrically formed with a distal end of the barrel 11. The cylinder 15 disposed in the tip 12. A internal threads 151 are formed on an inner surface of the tip 12 and are adapted to threadedly secure the protuberances 142 of the needle assembly 14 when the needle assembly 14 and the casing tube 11 are assembled.

The locking cover assembly 20 includes a grooved body 21 having an extension 210, a pivot hole 212 through an end of the extension 210, two opposite protrusions 214 on an inner surface at the end of the extension 210, and a projection 213 formed on one surface of the grooved body 21 and the extension 210 as a handle; and a groove 22 formed in the grooved body 21; and first and second spaced locking members 221, 222 formed on two side surfaces of the groove 22 respectively.

The pivot assembly 30 includes a base 34 mounted on the circumferential surface of the tip 12, the base 34 having first and second stops 341, 342 complimentarily engaged and formed on a rear end, a cylindrical neck 33 extending outward from the base 34, an enlarged latch 31 formed on an end of the neck 33, and a slot 32 formed on the latch 31. The pivot hole 212 is put through the latch 31 to pivotably secure the locking cover assembly 20 to the neck 33, i.e., put on the neck 33.

In a ready to administer injections state, the locking cover assembly 20 is perpendicular to the barrel 10 (see FIG. 1). Also, one protrusion 214 engages the first and second stops 341, 342. After use, a medical employee may turn the locking cover assembly 20 counterclockwise about 90 degrees by holding and pivoting the projection 213 until the other protrusion 214 is stopped by the first and second stops 341, 342. Also, the needle 141 passes the first locking member 221 to be locked by both the first and second locking members 221, 222. The used needle 141 is concealed in the groove 22 and can prevent the needle 141 from accidentally pricking the medical employee to contaminate the medical employee with microorganisms or blood on the needle 141. Further, this can prevent the safety syringe from being used for a second time. Thus, the safety syringe is ready for disposal.

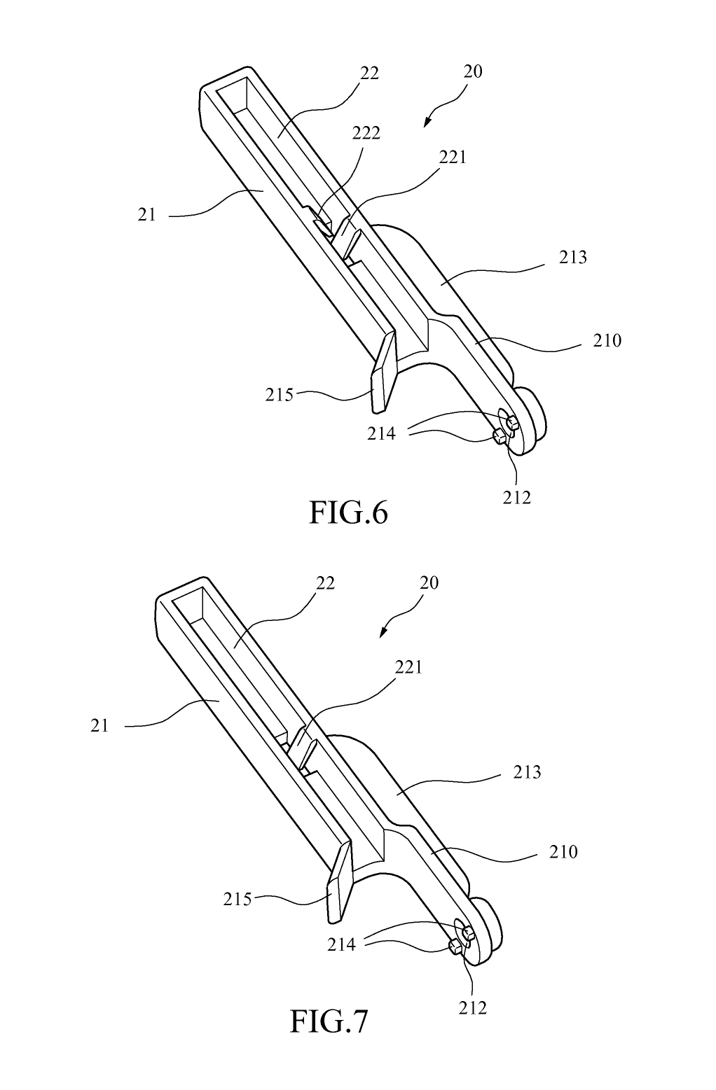

As shown in FIG. 6 specifically, in a second configuration of the locking cover assembly 20 is further provided with a splay member 215 proximate the first and second locking member 221, 222. The splay member 215 provide a wider space to allow the needle 141 to be correctly inserted into the groove 22.

As shown in FIG. 7 specifically, in another configuration the second locking member 222 is eliminated without compromising the characteristic of locking the needle 141 after use.

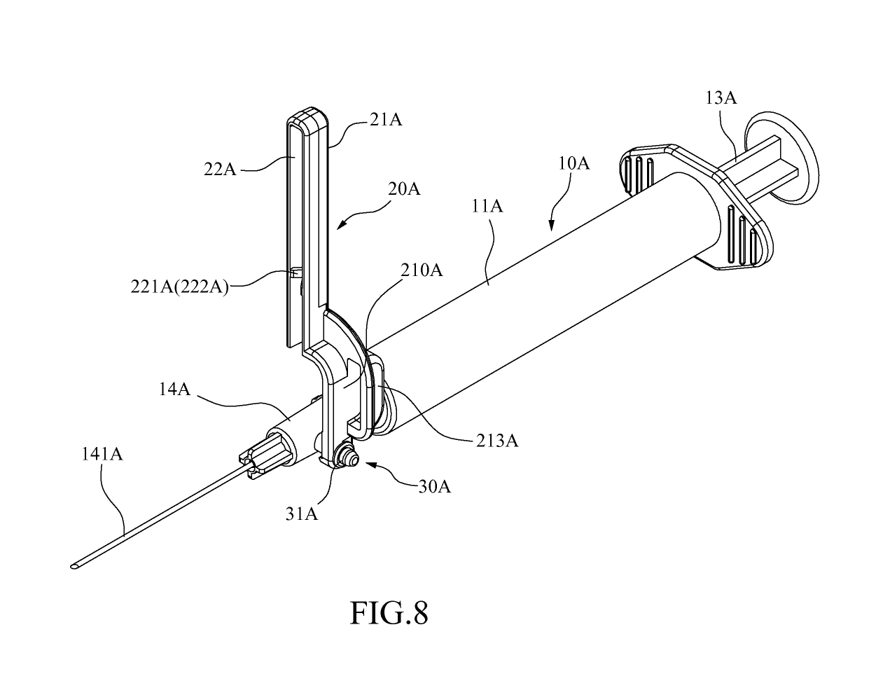

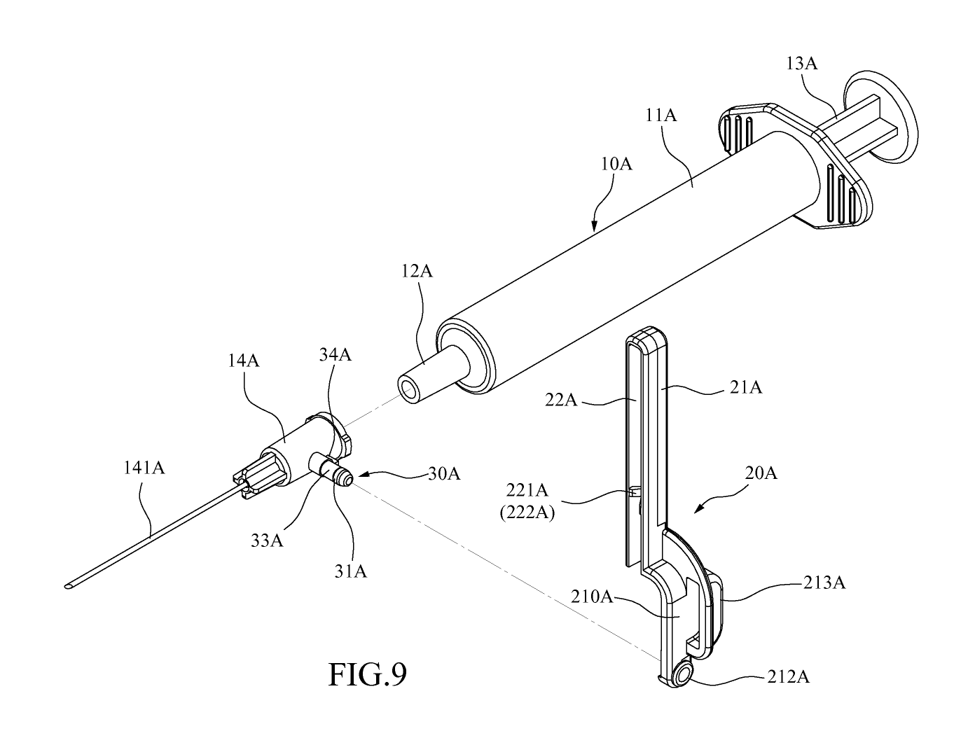

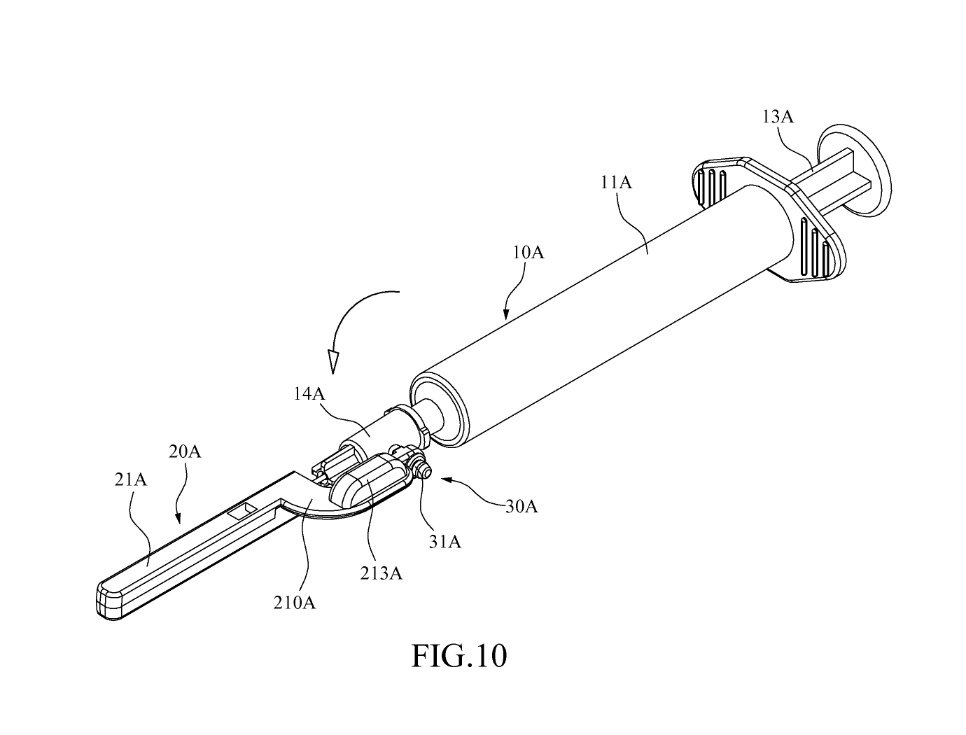

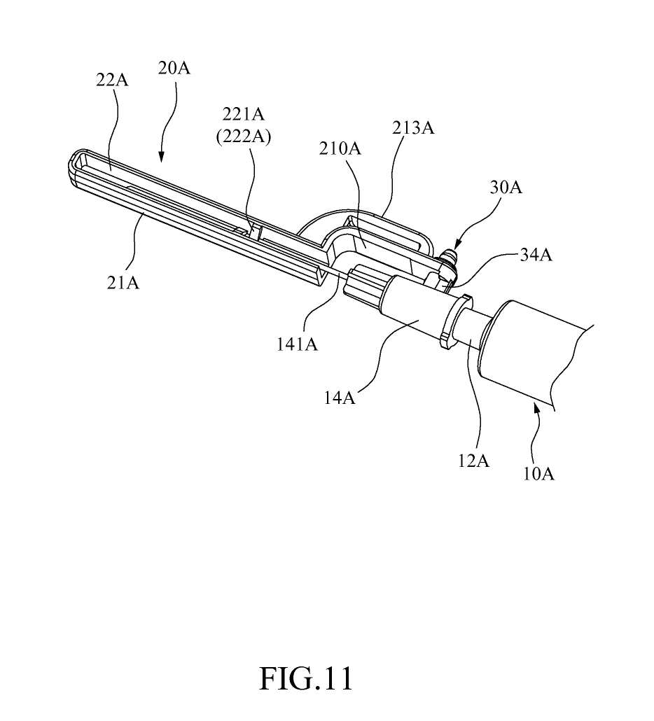

Referring to FIGS. 8 to 11, a safety syringe in accordance with a second preferred embodiment of the invention comprises a barrel 10A, a locking cover assembly 20A, and a pivot assembly 30A as discussed in detail below.

The barrel 10A includes a cylindrical casing tube 11A, a cylindrical tip 12A having a diameter less than that of the casing tube 11A, and a stem 13A. A needle assembly 14A is mounted to the tip 12A and has a hypodermic needle 141A. The pivot assembly 30A is implemented as a stepped-diameter shaft and includes a base 34A formed with an outer surface of the needle assembly 14A, an enlargement 31A at an end, and a cylindrical neck 33A having a diameter less than that of each of the base 34A and the enlargement 31A.

The locking cover assembly 20A includes a grooved body 21A having an extension 210A, a pivot hole 212A at an end of the extension 210A, an arc member 213A formed on the extension 210A as a handle; a groove 22A formed in the grooved body 21A; and first and second locking members 221A, 222A complementarily formed on two side surfaces of the groove 22A respectively. The needle assembly 14A further includes a front needle 141A. The tip 12A of the casing tube 11A is releasably fastened in the needle assembly 14A.

The pivot assembly 30A is inserted through the pivot hole 212A. The pivot hole 212A is put on the neck 33A after passing the enlargement 31A and being stopped by the base 34A. Thus, the pivot assembly 30A and the needle assembly 14A are pivotably assembled.

The pivot assembly 30A is adapted to pivot rearward about the neck 33A or after use, pivot forward to cause the needle 141A to dispose between the first and second locking member 221A, 222A to be locked by both the first and second locking members 221A, 222A.

The used needle 141A is concealed in the groove 22A. This can prevent the needle 141A from accidentally pricking the medical employee to contaminate the medical employee with microorganisms or blood on the needle 141A. Further, this can prevent the safety syringe from being used for a second time. Thus, the safety syringe is ready for disposal.

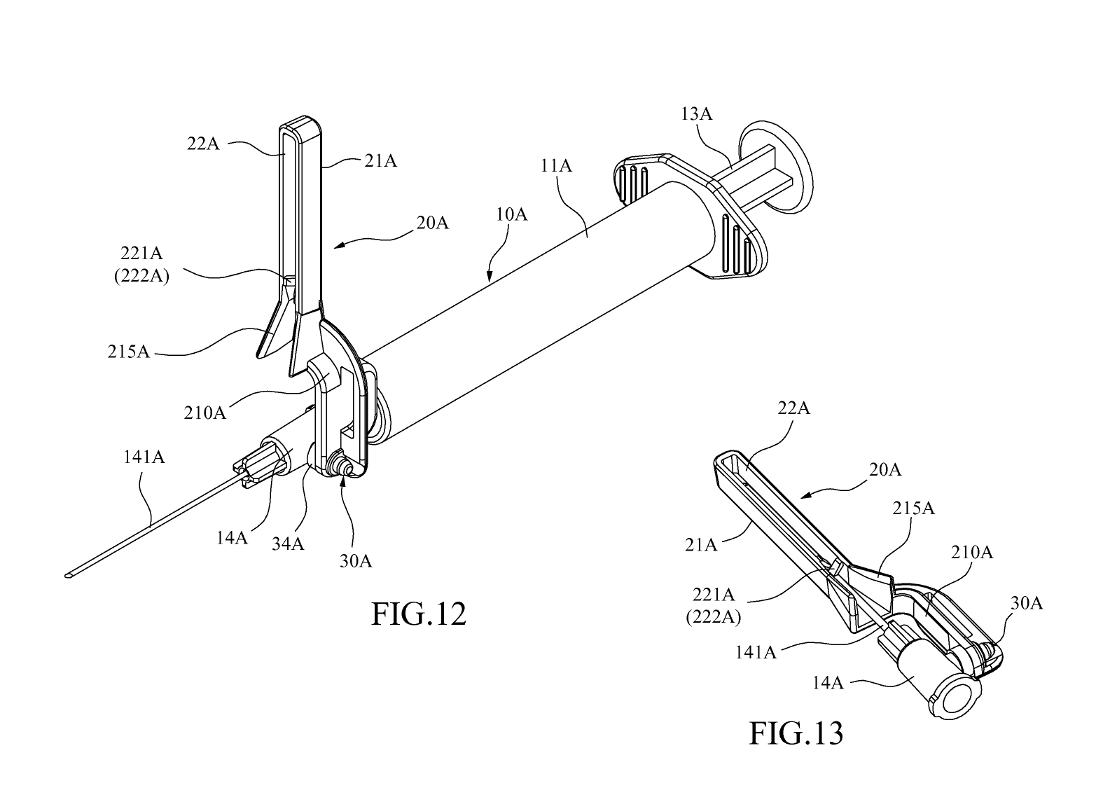

Referring to FIGS. 12 and 13, in a second configuration of the locking cover assembly 20A is further provided with two opposite splay members 215A proximate the first and second locking member 221A, 222A. The splay members 215A provide a wider space to allow the needle 141A to be correctly inserted into the groove 22A.

The second locking member 222A is eliminated without compromising the characteristic of locking the needle 141A after use.

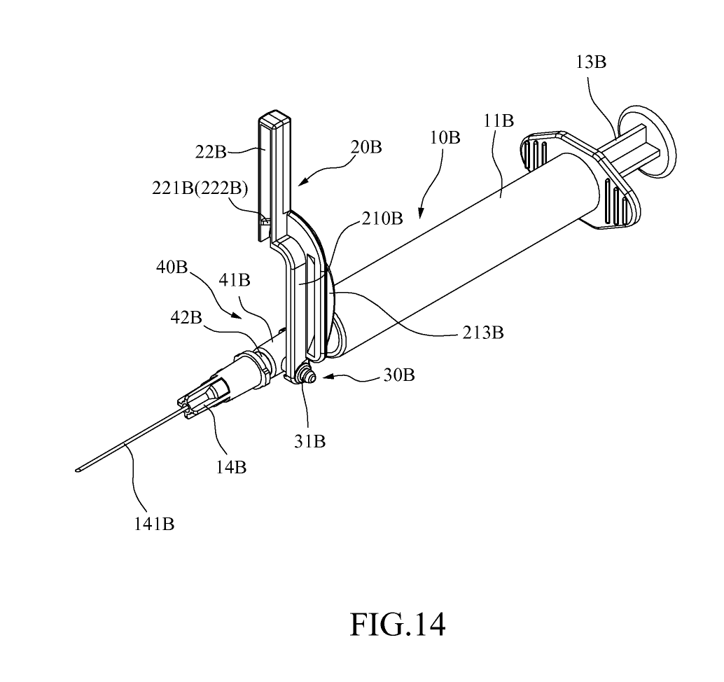

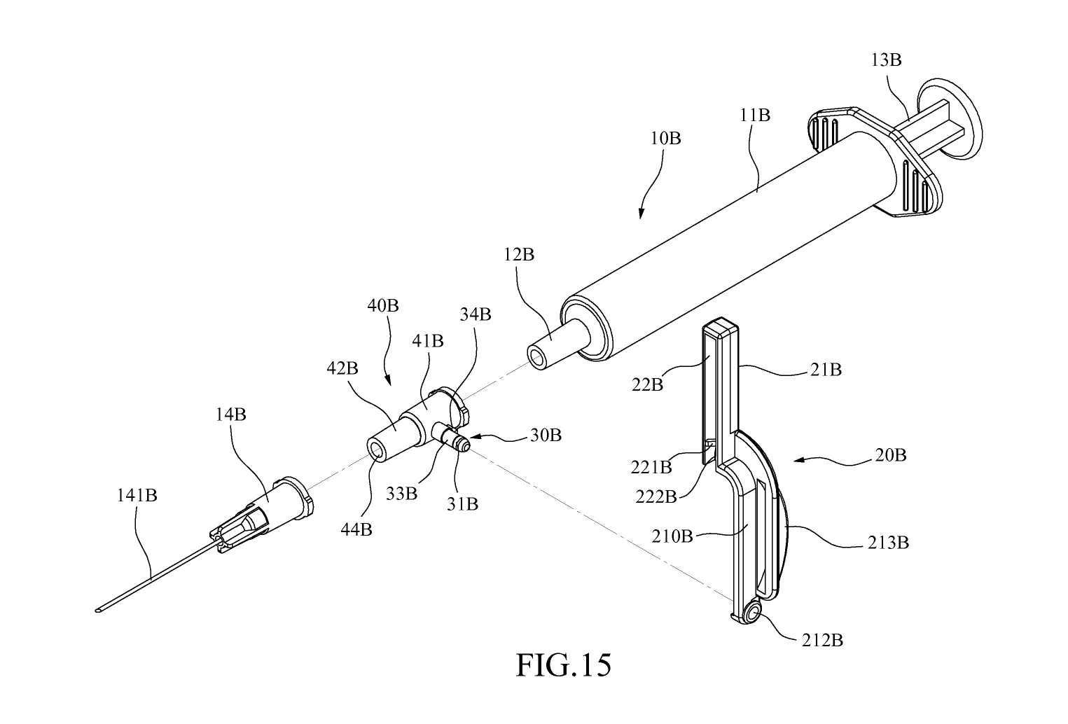

Referring to FIGS. 14 to 16, a safety syringe in accordance with a third preferred embodiment of the invention comprises a barrel 10B, a locking cover assembly 20B, a pivot assembly 30B, and a sleeve shaped adapter 40B as discussed in detail below.

The barrel 10B includes a cylindrical casing tube 11B, a cylindrical tip 12B having a diameter less than that of the casing tube 11B, and a stem 13B. A needle assembly 14B has a hypodermic needle 141B.

The sleeve shaped adapter 40B includes a central bore 44B, a larger diameter section 41B proximate the barrel 11B, a smaller diameter section 42B with the needle assembly 14B lockingly put thereon.

The pivot assembly 30B having a base 34B formed on an outer surface of the larger diameter section 41B, an enlargement 31B at an end, and a neck 33B between the base 34B and the enlargement 31B.

The locking cover assembly 20B includes a pivot hole 212B at an end of the extension 210B. An arc member 213B is formed on one surface of the extension 210B. The tip 12B is lockingly disposed in the adapter 40B. The pivot hole 212B is pivotably put on the neck 33B after passing the enlargement 31B and being stopped by the base 34B.

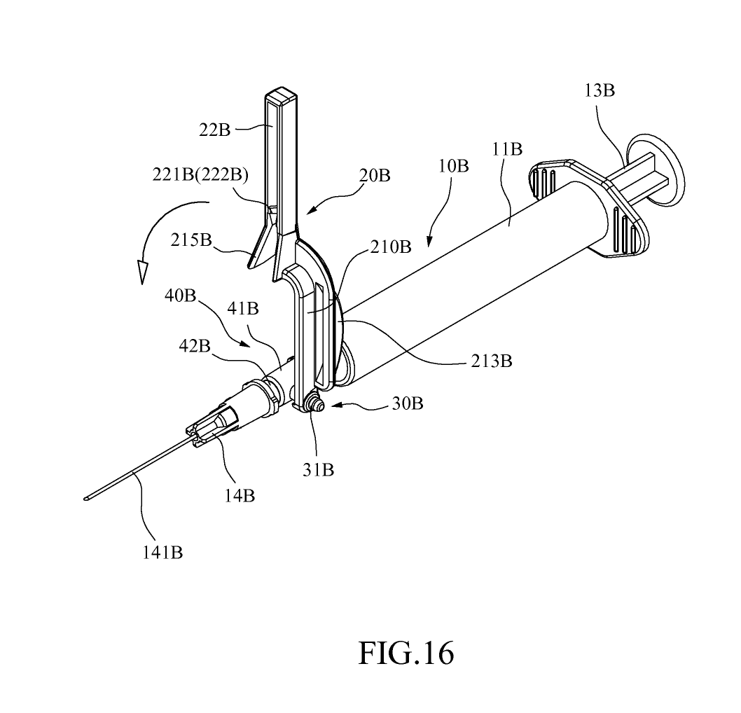

As shown in FIG. 16 specifically, in a second configuration of the locking cover assembly 20B is further provided with two opposite splay members 215B proximate the first and second locking member 221B, 222B. The splay members 215B provide a wider space to allow the needle 141B to be correctly inserted into the groove 22B.

The second locking member 222B is eliminated without compromising the characteristic of locking the needle 141B after use.

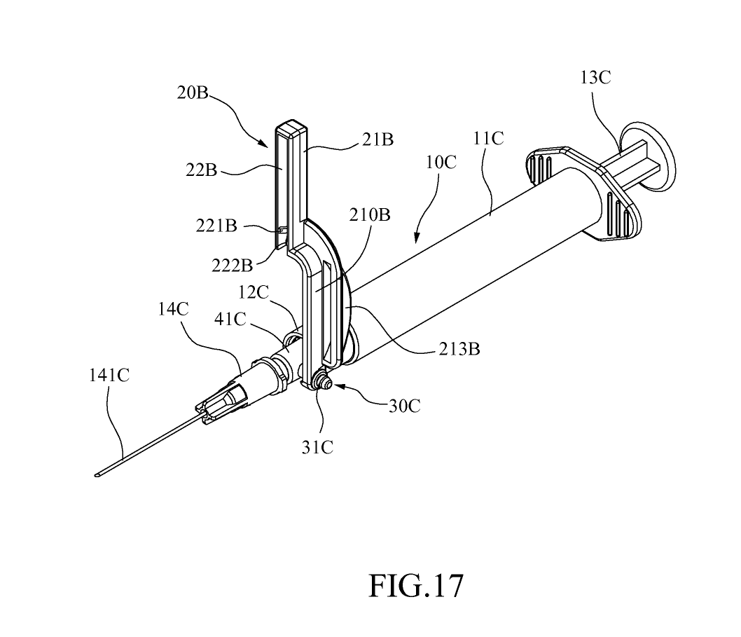

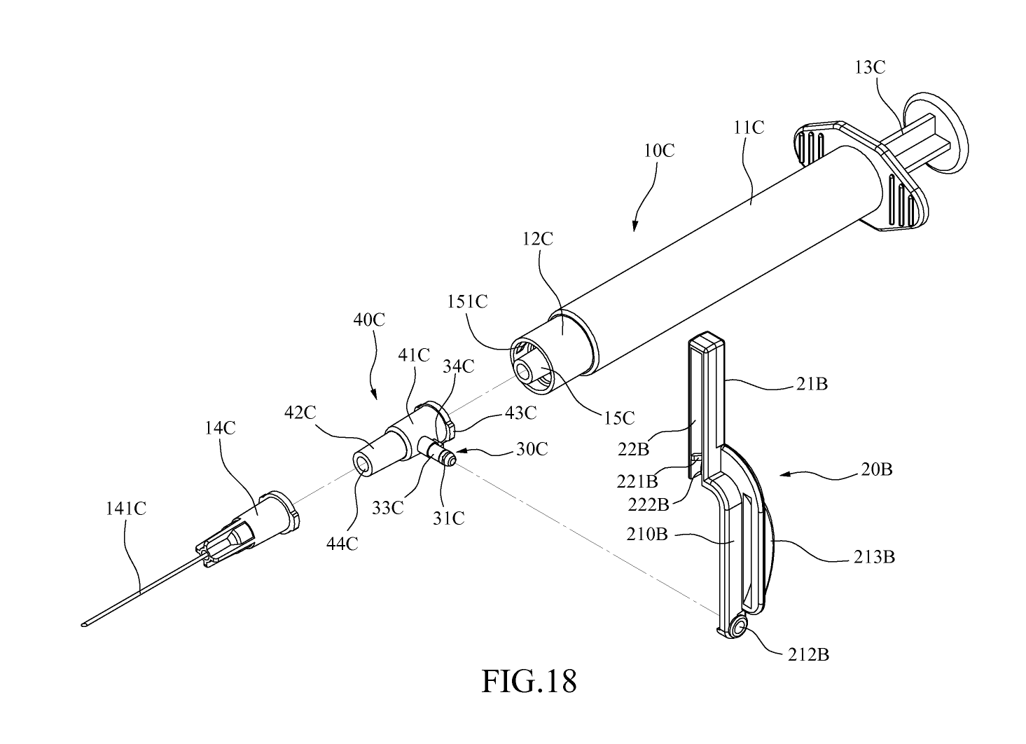

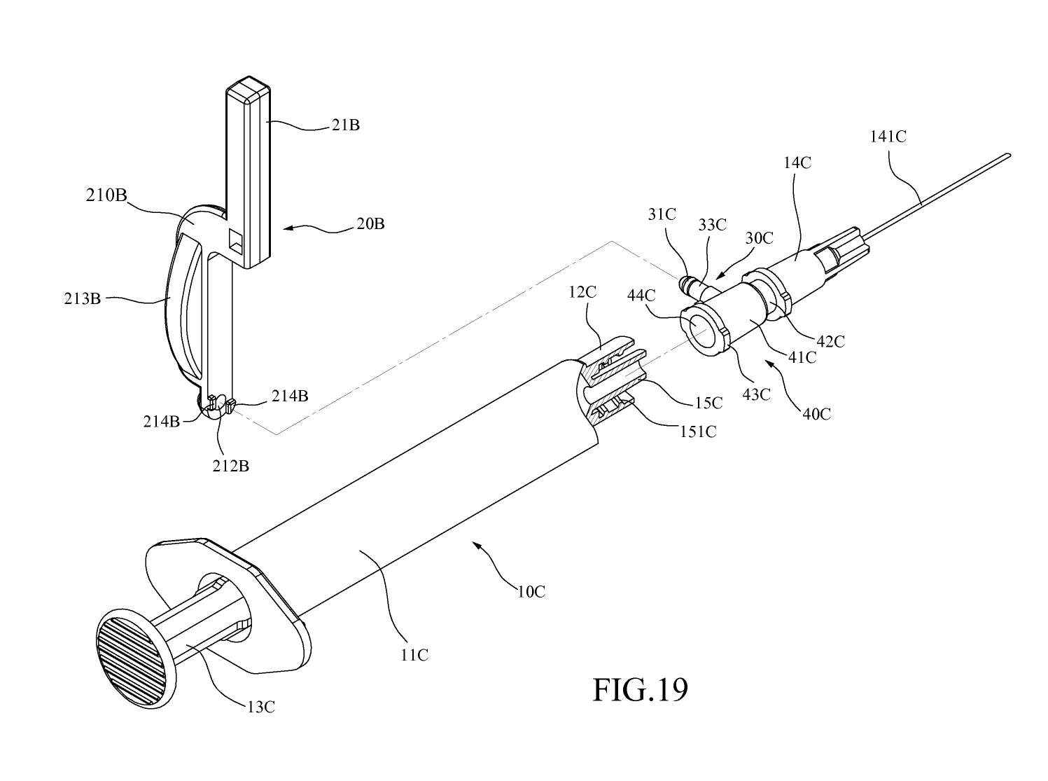

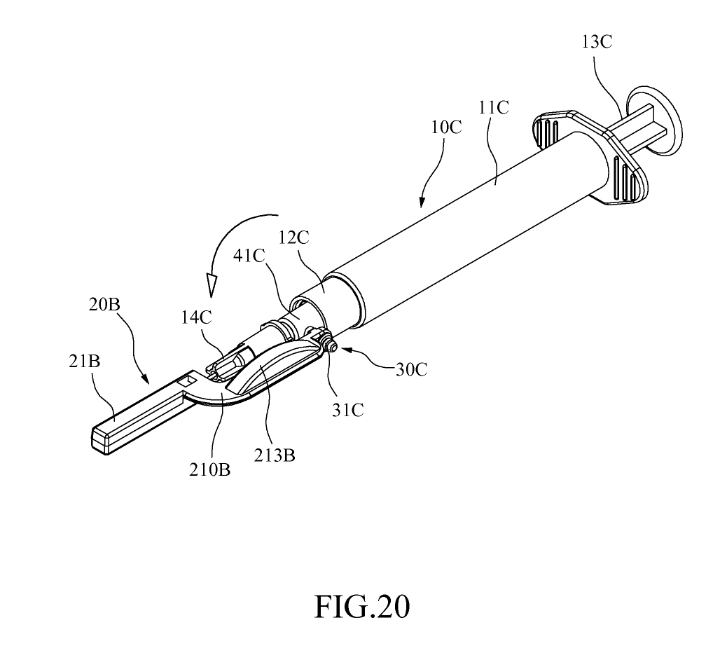

Referring to FIGS. 17 to 22, a safety syringe in accordance with a fourth preferred embodiment of the invention comprises a barrel 10C, a locking cover assembly 20B, a pivot assembly 30C, and a sleeve shaped adapter 40C as discussed in detail below.

The barrel 10C includes a cylindrical casing tube 11C, a cylindrical tip 12C having a diameter less than that of the casing tube 11C, and a stem 13C. A needle assembly 14C has a hypodermic needle 141C.

The sleeve shaped adapter 40C includes a central bore 44C, a larger diameter section 41C proximate the barrel 11C, two opposite protuberances 43C formed on a rear end of the larger diameter section 41C, a smaller diameter section 42C with the needle assembly 14C lockingly put thereon.

The tip 12C is enlarged and a hollow cylinder 15C is concentrically formed with a distal end of the barrel 11C. The cylinder 15C is disposed in the tip 12C. Internal threads 151C are formed on an inner surface of the tip 12C and are adapted to threadedly secure the protuberances 43C when the larger diameter section 41C is put on the cylinder 15C.

The pivot assembly 30C having a base 34C formed on an outer surface of the larger diameter section 41C, an enlargement 31C on an end, and a neck 33C between the base 34C and the enlargement 31C.

The locking cover assembly 20B includes a pivot hole 212B at an end of the extension 210B. An arc member 213B is formed on one surface of the extension 210B. The pivot hole 212B is pivotably put on the neck 33C after passing the enlargement 31C and being stopped by the base 34C.

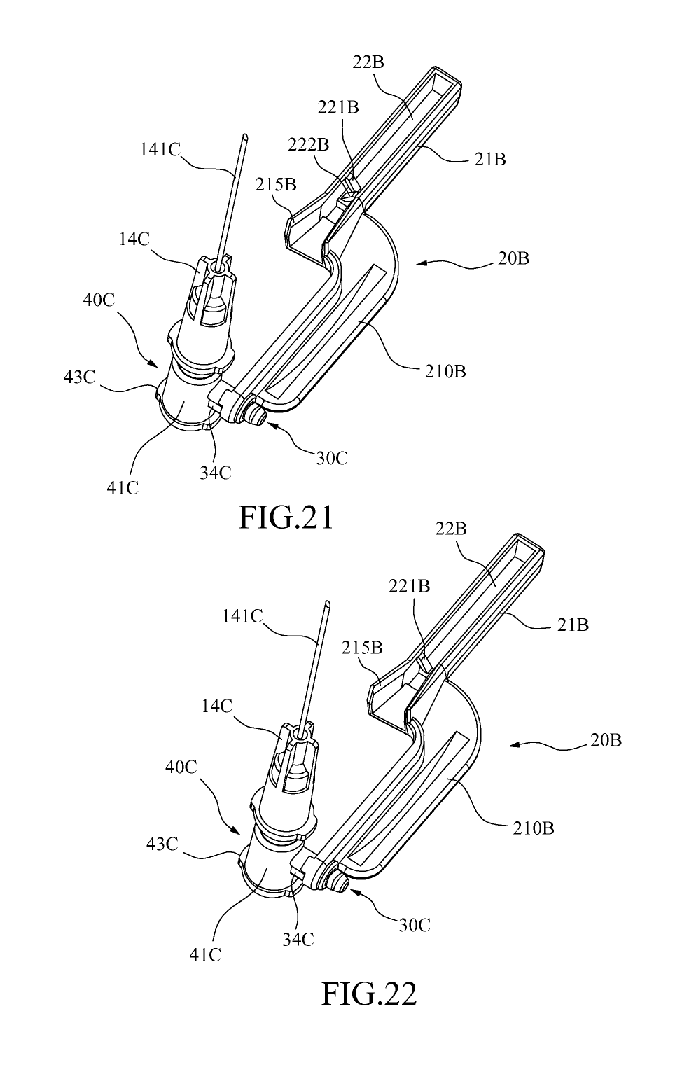

As shown in FIG. 21, in a second configuration of the locking cover assembly 20B is further provided with two opposite splay members 215B proximate the first and second locking member 221B, 222B. The splay members 215B provide a wider space to allow the needle 141C to be correctly inserted into the groove 22B.

As shown in FIG. 22 specifically, in a second configuration the second locking member 222B is eliminated without compromising the characteristic of locking the needle 141C after use.

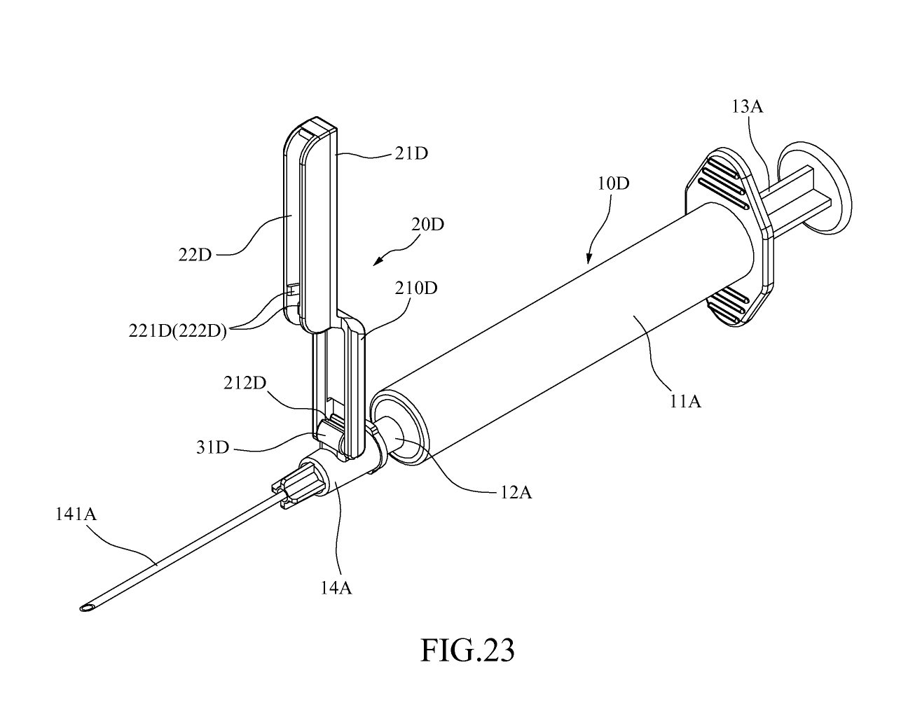

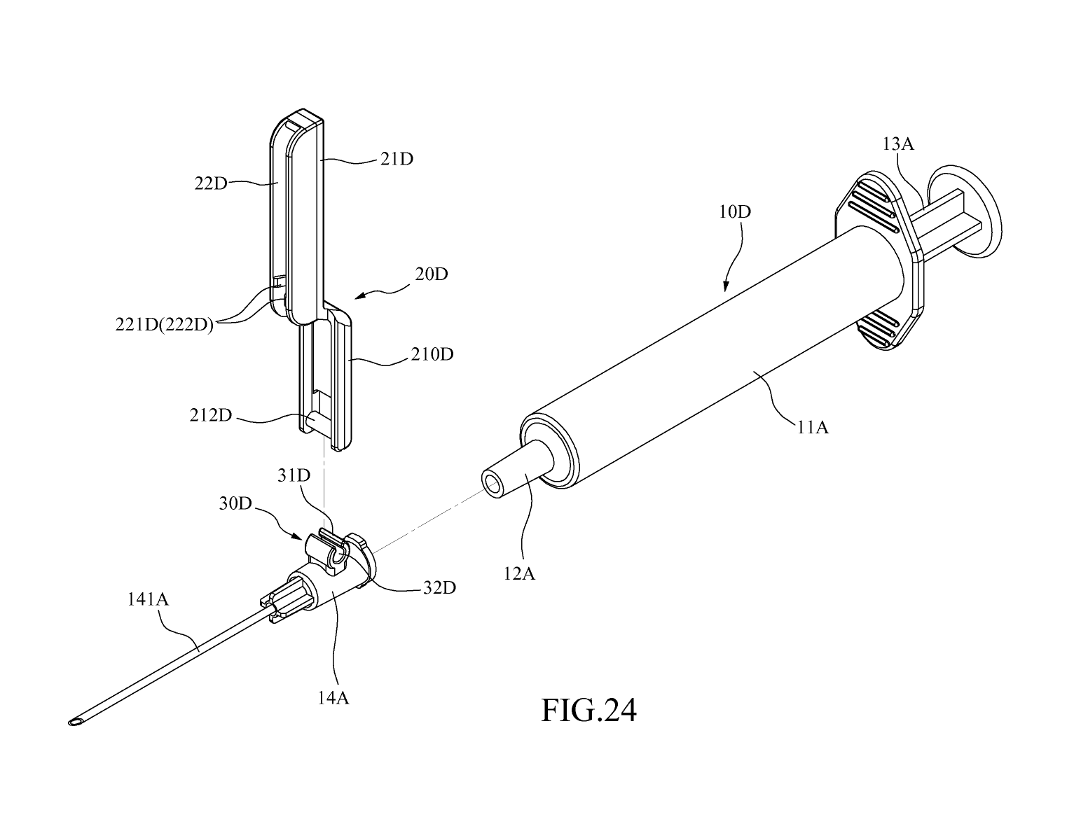

Referring to FIGS. 23 to 25, a safety syringe in accordance with a fifth preferred embodiment of the invention comprises a barrel 10D, a locking cover assembly 20D, and a pivot assembly 30D as discussed in detail below.

The barrel 10D is substantially the same as the barrel 10A of the second preferred embodiment except the following:

The pivot assembly 30D having a C-shaped longitudinal section is formed with the needle assembly 14A having a needle 141A. The pivot assembly 30D has two ends open and includes a central trough 32D having a top opening 31D.

The locking cover assembly 20D includes a grooved body 21D having an extension 210D, shaft 212D formed across two sides of an inner surface at an end of the extension 210D; and a groove 22D formed in the grooved body 21D; and first and second spaced locking members 221D, 222D formed on two side surfaces of the groove 22D respectively.

The shaft 212D may pass through the opening 31D to pivotably dispose in the trough 32D.

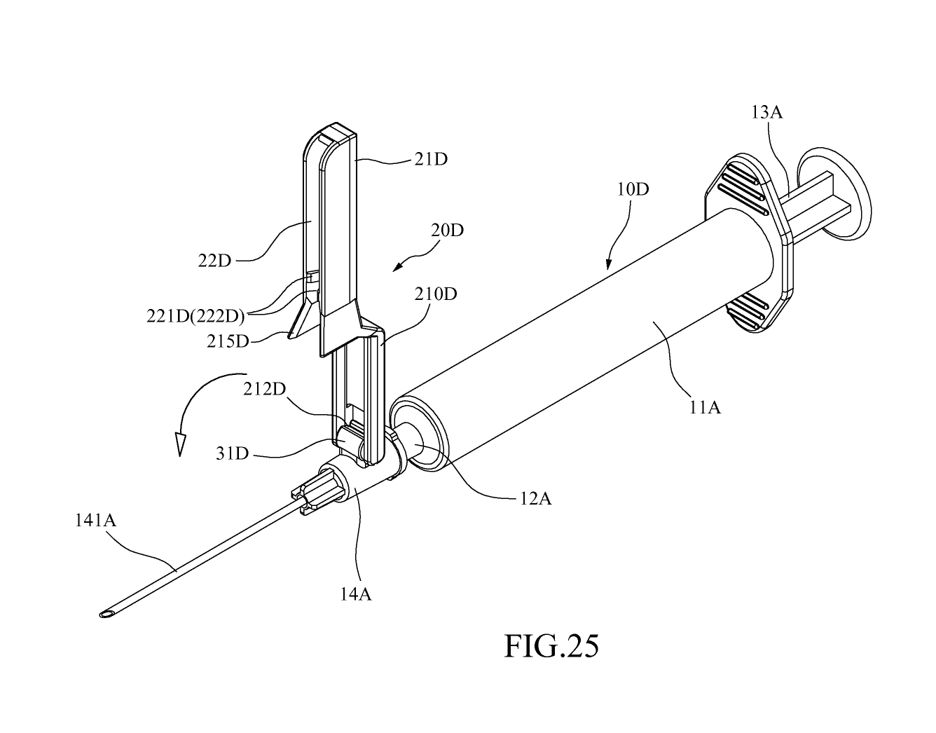

As shown in FIG. 25 specifically, in a second configuration of the locking cover assembly 20D is further provided with two opposite splay members 215D proximate the first and second locking member 221D, 222D. The splay members 215D provide a wider space to allow the needle 141A to be correctly inserted into the groove 22D.

The second locking member 222D is eliminated without compromising the characteristic of locking the needle 141A after use.

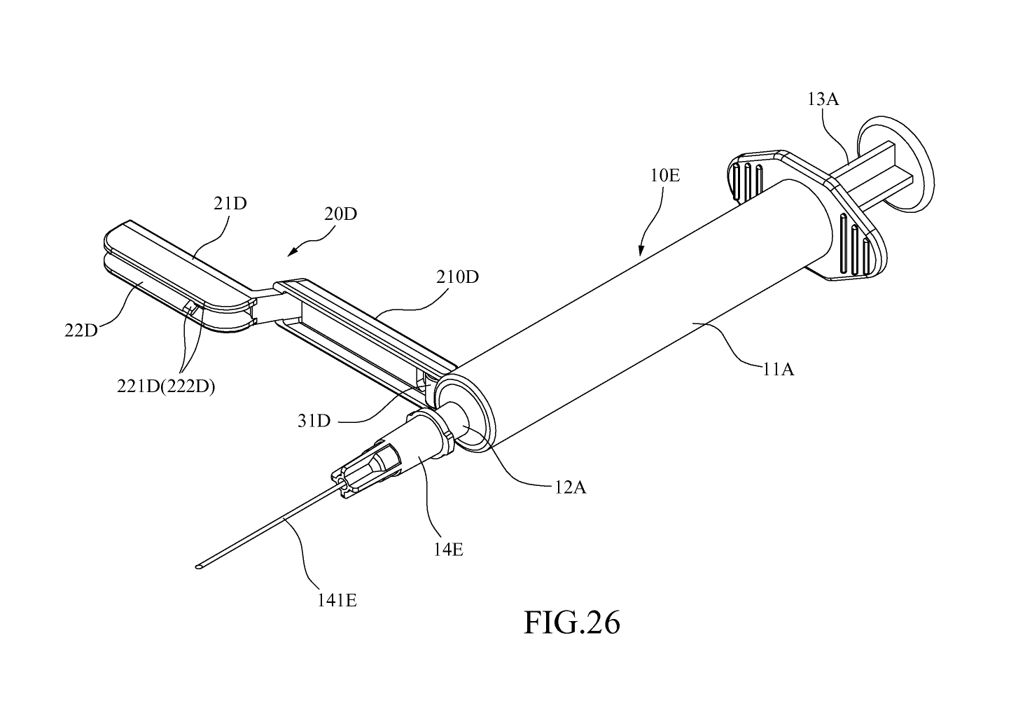

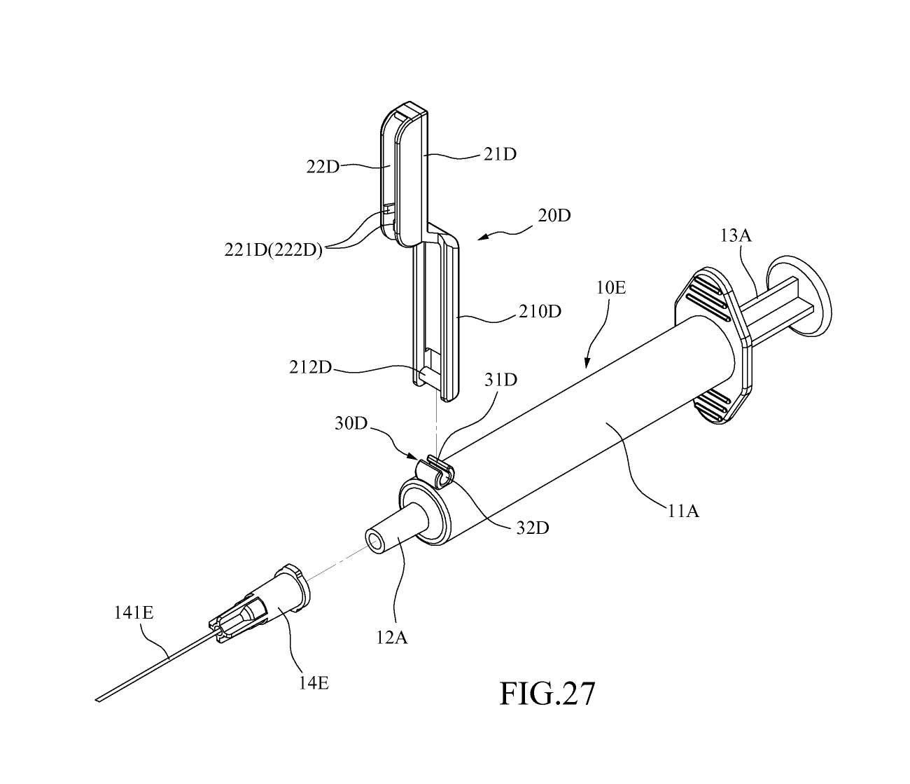

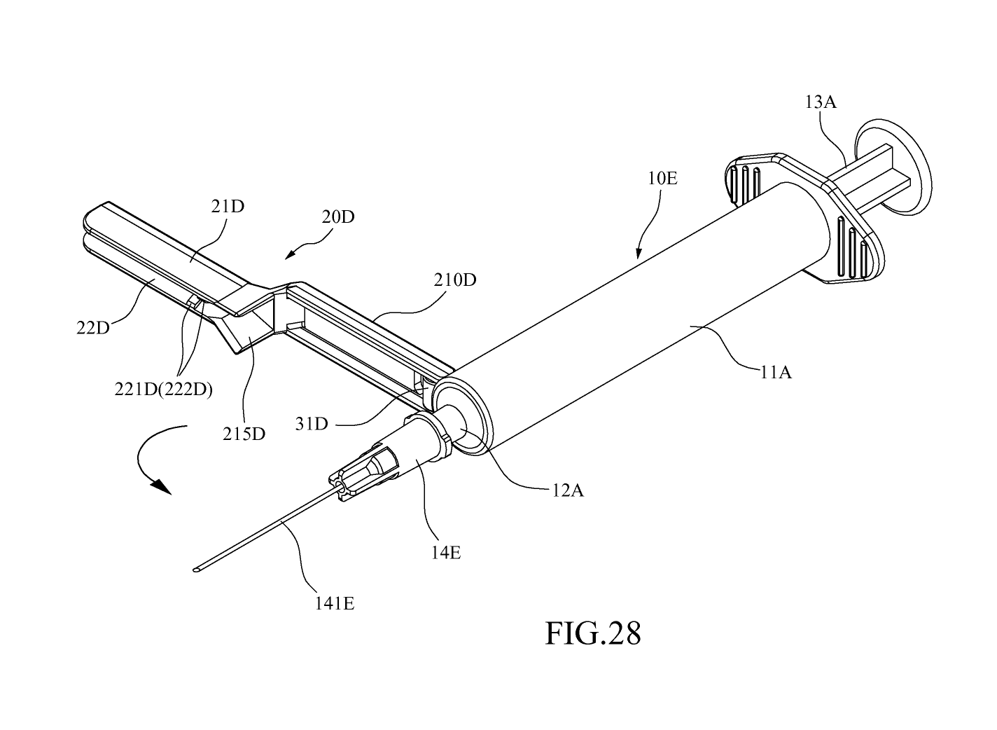

Referring to FIGS. 26 to 28, a safety syringe in accordance with a sixth preferred embodiment of the invention comprises a barrel 10E, a locking cover assembly 20D, and a pivot assembly 30D as discussed in detail below.

The characteristics of the sixth preferred embodiment are substantially the same as that of the fifth preferred embodiment except the following:

The pivot assembly 30D having a C-shaped longitudinal section is formed with the casing tube 11A. The needle assembly 14E having a needle 141E is lockingly put on the tip 12A.

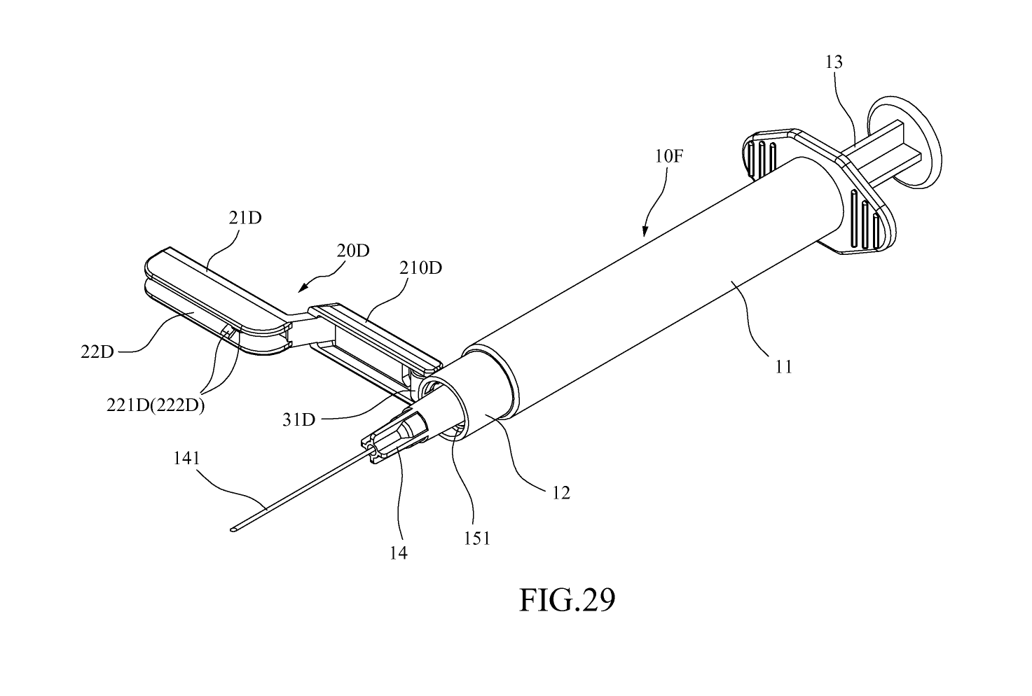

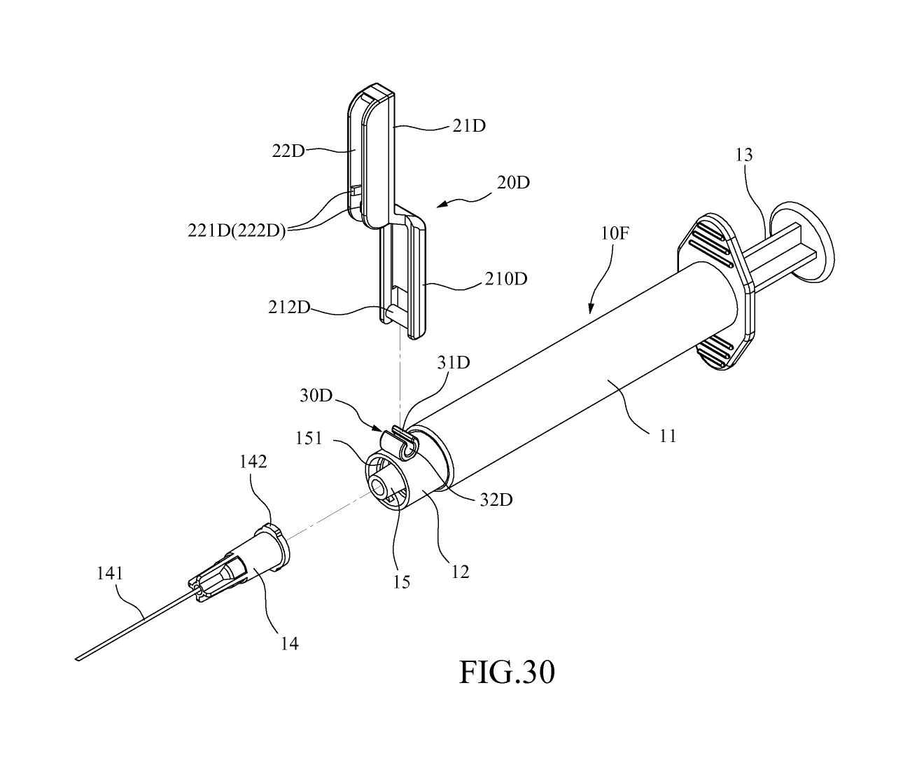

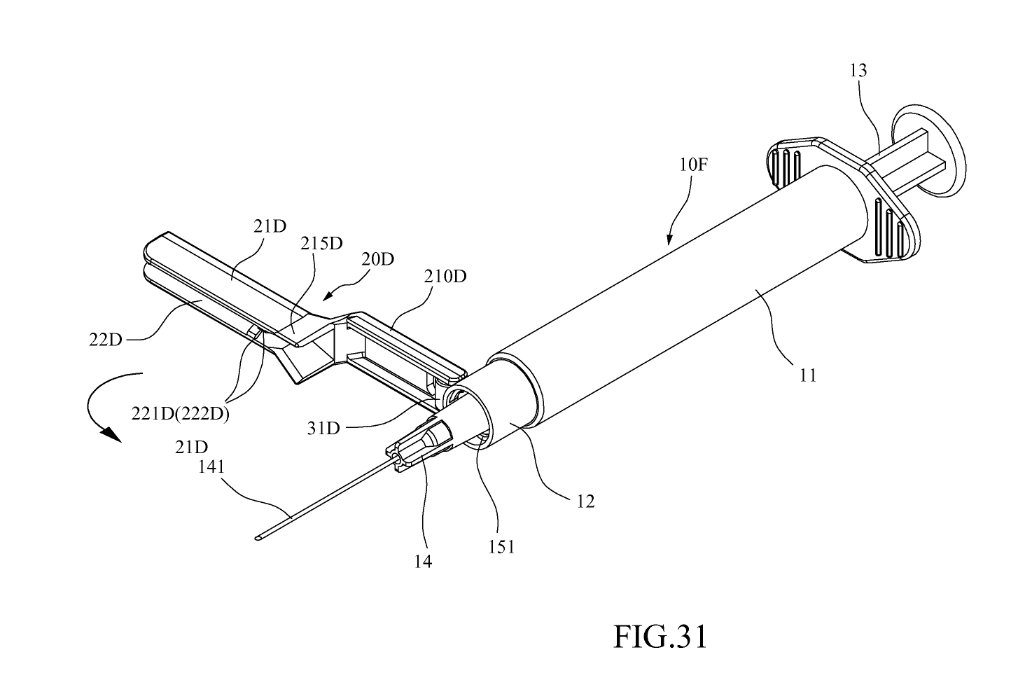

Referring to FIGS. 29 to 31, a safety syringe in accordance with a seventh preferred embodiment of the invention comprises a barrel 10F, a locking cover assembly 20D, and a pivot assembly 30D as discussed in detail below.

The characteristics of the seventh preferred embodiment are substantially the same as that of the first preferred embodiment except the following:

The pivot assembly 30D having a C-shaped longitudinal section is formed with the tip 12 of the barrel 10F, and the shaft 212D of the pivot assembly 30D may pass through the opening 31D to pivotably dispose in the trough 32D.

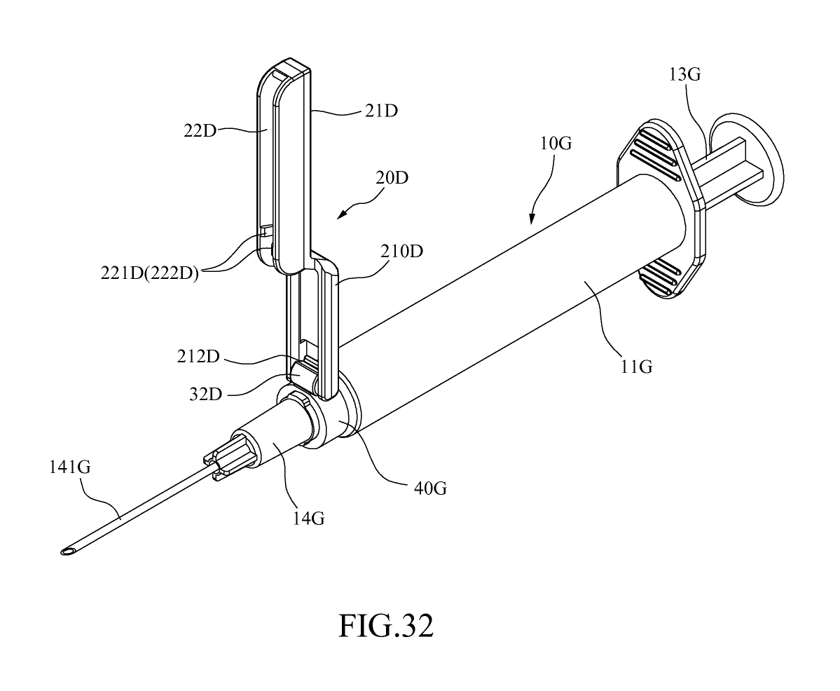

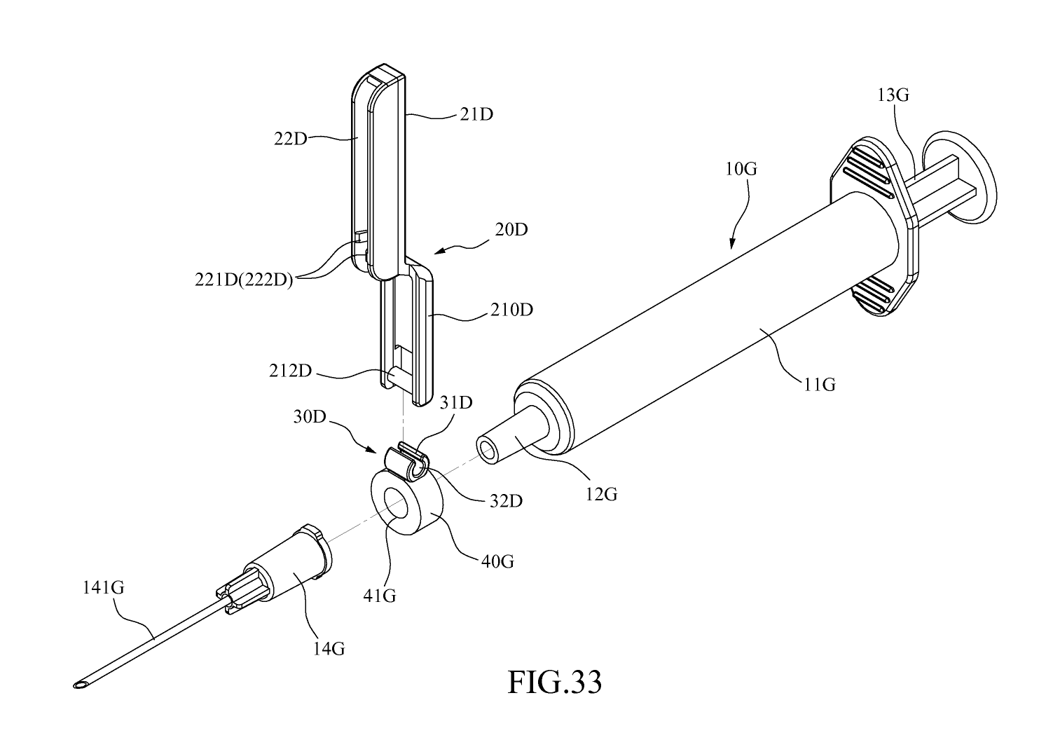

Referring to FIGS. 32 to 34, a safety syringe in accordance with an eighth preferred embodiment of the invention comprises a barrel 10G, a locking cover assembly 20D, a pivot assembly 30D, and a sleeve shaped adapter 40G as discussed in detail below.

The barrel 10G includes a cylindrical casing tube 11G, a cylindrical tip 12G having a diameter less than that of the casing tube 11G, and a stem 13G. A needle assembly 14G has a hypodermic needle 141G.

A hollow, cylindrical adapter 40G includes an axial channel 41G. The tip 12G is inserted through the channel 41G into the needle assembly 14G to fasten the casing tube 11G and the needle assembly 14G together. The pivot assembly 30D is formed on a periphery of the adapter 40G and has two ends open. The pivot assembly 30D includes a central trough 32D having a top opening 31D. The shaft 212D may pass through the opening 31D to pivotably dispose in the trough 32D. The shaft 212D may pass through the opening 31D to pivotably dispose in the trough 32D.

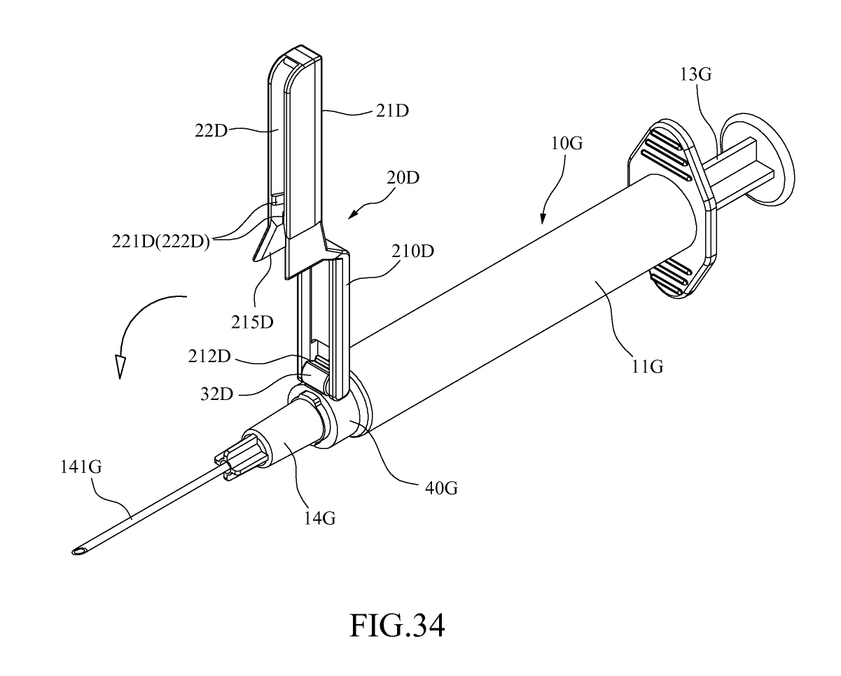

As shown in FIG. 34 specifically, in a second configuration of the locking cover assembly 20D is further provided with two opposite splay members 215D proximate the first and second locking member 221D, 222D. The splay members 215D provide a wider space to allow the needle 141A to be correctly inserted into the groove 22D.

The second locking member 222D is eliminated without compromising the characteristic of locking the needle 141G after use.

While the invention has been described in terms of preferred embodiments, those skilled in the art will recognize that the invention can be practiced with modifications within the spirit and scope of the appended claims.

What is claimed is:

1. A safety syringe comprising:
a barrel (10, 10E, 10F) including a casing tube (11, 11A) and a hollow, cylindrical tip (12, 12A);
a needle assembly (14, 14E) releasably secured to the tip (12, 12A);
a pivot assembly (30, 30D) releasably secured to the barrel (10, 10E, 10F); and
a locking cover assembly (20, 20D) pivotably secured to the pivot assembly (30, 30D);
wherein in inoperative position, the locking cover assembly (20, 20D) is distal the needle assembly (14, 14E) which is exposed; and
wherein in response to a dispensing operation of the needle assembly (14, 14E), a pivotal movement of the locking cover assembly (20, 20D) toward the needle assembly (14, 14E) is configured to cover and lock the needle assembly (14, 14E).

2. The safety syringe of claim 1, wherein
the tip (12) includes internal threads (151) on an inner surface;
the needle assembly (14) includes two opposite protuberances (142) on a rear end, the protuberances (142) being threadedly secured to the threads (151) so as to fasten the needle assembly (14) and the tip (12) of the barrel (10) together;
the pivot assembly (30) is secured to the tip (12) and includes an enlarged latch (31) formed at an end of the neck (33), and a slot (32) formed on the latch (31); and
the locking cover assembly (20) is releasably secured to the neck (33).

3. The safety syringe of claim 2, wherein the locking cover assembly (20) includes a grooved body (21) having an extension (210), and a pivot hole (212) through an end of the extension (210); a groove (22) formed in the grooved body (21); and at least one of first and second locking members (221, 222) formed on at least one side of the groove (22); and wherein the pivot hole (212) is pivotably fastened on the neck (33).

4. The safety syringe of claim 3, further comprising with a splay member (215) at an end of the groove (22) proximate the extension (210).

5. The safety syringe of claim 1, wherein the needle assembly (14E) is mounted on the tip (12A); wherein the pivot assembly (30D) is mounted on a periphery of a front end of the casing tube (11A) and includes a central trough (32D) having a C-shaped longitudinal section and an opening (31D) distal the casing tube (11A); and wherein the locking cover assembly (20D) is pivotably secured to the central trough (32D).

6. The safety syringe of claim 5, wherein the locking cover assembly (20D) includes a grooved body (21D) having an extension (210D), a shaft (212D) formed across two sides of an inner surface at an end of the extension (210D), a groove (22D) formed in the grooved body (21D), and at least one of first and second locking members (221D, 222D) formed on at least one side of the groove (22D); and wherein the shaft (212D) is pivotably fastened in the trough (32D).

7. The safety syringe of claim 6, further comprising with two opposite splay members (215D) at an end of the groove (22D) proximate the extension (210D).

8. The safety syringe of claim 1, wherein
the tip (12) includes internal threads (151) on an inner surface;
the needle assembly (14) includes two opposite protuberances (142) on a rear end, the protuberances (142) being threadedly secured to the threads (151) so as to fasten the needle assembly (14) and the tip (12) of the barrel (10F) together;
the pivot assembly (30D) is secured to the tip (12) and includes a central trough (32D) having a C-shaped longitudinal section and an opening (31D) distal the casing tube (11); and
the locking cover assembly (20D) is pivotably secured to the central trough (32D).

9. The safety syringe of claim 8, wherein the locking cover assembly (20D) includes a grooved body (21D) having an extension (210D), a shaft (212D) formed across two sides of an inner surface at an end of the extension (210D), a groove (22D) formed in the grooved body (21D), and at least one of first and second locking members (221D, 222D) formed on at least one side of the groove (22D); and wherein the shaft (212D) is pivotably fastened in the trough (32D).

10. The safety syringe of claim 9, further comprising with two opposite splay members (215D) at an end of the groove (22D) proximate the extension (210D).

11. A safety syringe comprising:
a barrel (10A, 10D) including a casing tube (11A) and a hollow, cylindrical tip (12A);
a needle assembly (14A) releasably secured to the tip (12A);
a pivot assembly (30A, 30D) secured to the needle assembly (14A); and
a locking cover assembly (20A, 20D) pivotably secured to the pivot assembly (30A, 30D);
wherein in inoperative position, the locking cover assembly (20A, 20D) is distal the needle assembly (14A) which is exposed; and
wherein in response to a dispensing operation of the needle assembly (14A), a pivotal movement of the locking cover assembly (20A, 20D) toward the needle assembly (14A) is configured to cover and lock the needle assembly (14A).

12. The safety syringe of claim 11, wherein the pivot assembly (30A) includes a base (34A) formed with the needle assembly (14A), an enlargement (31A) at an end, and a cylindrical neck (33A) having a diameter less than that of each of the base (34A) and the enlargement (31A); and wherein the locking cover assembly (20A) is releasably secured to the neck (33A).

13. The safety syringe of claim 12, wherein the locking cover assembly (20A) includes a grooved body (21A) having an extension (210A), and a pivot hole (212A) through an end of the extension (210A); a groove (22A) formed in the grooved body (21A); and at least one of first and second locking members (221A, 222A) formed on at least one side of the groove (22A); and wherein the pivot hole (212A) is pivotably fastened on the neck (33A) by passing through the enlargement (31A).

14. The safety syringe of claim 13, further comprising with two opposite splay members (215A) at an end of the groove (22A) proximate the extension (210A).

15. The safety syringe of claim 11, wherein the pivot assembly (30D) includes a central trough (32D) having a C-shaped longitudinal section and an opening (31D) distal the casing tube (11A); and wherein the locking cover assembly (20D) is pivotably secured to the central trough (32D).

16. The safety syringe of claim 15, wherein the locking cover assembly (20D) includes a grooved body (21D) having an extension (210D), a shaft (212D) formed across two sides of an inner surface at an end of the extension (210D), a groove (22D) formed in the grooved body (21D), and at least one of first and second locking members (221D, 222D) formed on at least one side of the groove (22D); and wherein the shaft (212D) is pivotably fastened in the trough (32D).

17. The safety syringe of claim 16, further comprising with two opposite splay members (215D) at an end of the groove (22D) proximate the extension (210D).

18. A safety syringe comprising:
a barrel (10B, 10C, 10G) including a casing tube (11B, 11C, 11G) and a hollow, cylindrical tip (12B, 12C, 12G);
an adapter (40B, 40C, 40G) releasably secured to the tip (12B, 12C, 12G);
a needle assembly (14B, 14C, 14G) releasably secured to the adapter (40B, 40C, 40G);
a pivot assembly (30B, 30C, 30D) secured to the adapter (40B, 40C, 40G); and
a locking cover assembly (20B, 20D) pivotably secured to the adapter (40B, 40C, 40G);
wherein in inoperative position, the locking cover assembly (20B, 20D) is distal the needle assembly (14B, 14C, 14G) which is exposed; and
wherein in response to a dispensing operation of the needle assembly (14B, 14C, 14G), a pivotal movement of the locking cover assembly (20B, 20D) toward the needle assembly (14B, 14C, 14G) is configured to cover and lock the needle assembly (14B, 14C, 14G).

19. The safety syringe of claim 18, wherein the adapter (40B) is hollow, elongated and includes a larger diameter section (41B) proximate the barrel (11B), and a smaller diameter section (42B) with the needle assembly (14B) lockingly put thereon; wherein the pivot assembly (30B) includes a base (34B) formed with the larger diameter section (41B), an enlargement (31B) at an end, and a neck (33B) between the base (34B) and the enlargement (31B); and wherein the locking cover assembly (20B) is releasably secured to the neck (33B).

20. The safety syringe of claim 19, wherein the locking cover assembly (20B) includes a grooved body (21B) having an extension (210B), and a pivot hole (212B) through an end of the extension (210B); a groove (22B) formed in the grooved body (21B); and at least one of first and second locking members (221B, 222B) formed on at least one side of the groove (22B); and wherein the pivot hole (212B) is pivotably fastened on the neck (33B).

21. The safety syringe of claim 20, further comprising with two opposite splay members (215B) at an end of the groove (22B) proximate the extension (210B).

22. The safety syringe of claim 18, wherein
the tip (12C) includes internal threads (151C) on an inner surface;
the adapter (40C) includes a larger diameter section (41C) proximate the barrel (11C), a smaller diameter section (42C) secured to the needle assembly (14C), and two opposite protuberances (43C) on a rear end, the protuberances (43C) being threadedly secured to the threads (151C) so as to fasten the adapter (40C) and the tip (12C) of the barrel (10C) together;
the pivot assembly (30C) includes a base (34C) formed with the larger diameter section (41C), an enlargement (31C) at an end, and a neck (33C) between the base (34C) and the enlargement (31C);
the smaller diameter section (42C) is formed with the needle assembly (14C); and
the locking cover assembly (20B) is releasably secured to the neck (33C).

23. The safety syringe of claim 22, wherein the locking cover assembly (20B) includes a grooved body (21B) having an extension (210B), and a pivot hole (212B) through an end of the extension (210B); a groove (22B) formed in the grooved body (21B); and at least one of first and second locking members (221B, 222B) formed on at least one side of the groove (22B); and wherein the pivot hole (212B) is pivotably fastened on the neck (33C).

24. The safety syringe of claim 23, further comprising with two opposite splay members (215B) at an end of the groove (22B) proximate the extension (210B).

25. The safety syringe of claim 18, wherein the adapter (40G) is hollow, cylindrical and fastened on the tip (12G); wherein the pivot assembly (30D) is mounted on a periphery of the adapter (40G) and includes a central trough (32D) having a C-shaped longitudinal section and an opening (31D) distal the casing tube (11G); and wherein the locking cover assembly (20D) is pivotably secured to the central trough (32D).

26. The safety syringe of claim 25, wherein the locking cover assembly (20D) includes a grooved body (21D) having an extension (210D), a shaft (212D) formed across two sides of an inner surface at an end of the extension (210D), a groove (22D) formed in the grooved body (21D), and at least one of first and second locking members (221D, 222D) formed on at least one side of the groove (22D); and wherein the shaft (212D) is pivotably fastened in the trough (32D).

27. The safety syringe of claim 26, further comprising with two opposite splay members (215D) at an end of the groove (22D) proximate the extension (210D).
